(19) United States
(12) Patent Application Publication
(10) Pub. No.: US 2005/0026275 A1
(43) Pub. Date: Feb. 3, 2005

(54) Device, system and method for receiving, processing and dispersing cells

(76) Inventor: Andrej Bahoric, Thornhill (CA)

Correspondence Address: Debra M. Parrish, Esq.;Parrish Law Offices, Suite 200, 615 Washington Rd., Pittsburgh, PA, 15228, US

(21) Appl. No.: 10/874,896

(22) Filed: Jun. 23, 2004

Related U.S. Application Data
(60) Provisional application No. 60/480,713, filed on Jun. 23, 2003.

(51) Int. Cl. C12M 1/34

(52) U.S. Cl. 435/287.1

(57) ABSTRACT

The present invention relates to a system and method which includes a means for sterilely receiving and handling tissue, a means for mechanically handling and separating the tissue, a means for enzymatically disaggregating the tissue, a means for holding processing enzymes, a means for mechanically and thermally processing tissue, a means for storing processed tissue, and a multi-channel cell sprayer for delivering cells derived therefrom to a variety of surfaces, a means for storing components in a sterile manner, and a method for using such sprayer. More specifically, the present invention relates to a system for harvesting tissue, processing tissue and delivering cells derived therefrom to large areas such as wound surfaces or wound dressings.

### RELATED APPLICATION

This application claims priority from U.S. Application No. 60/480,713 entitled, SYSTEM AND METHOD FOR RECEIVING, PROCESSING AND DISPERSING CELLS,” filed on Jun. 23, 2003.

### FIELD OF THE INVENTION

This invention relates to a device, system and method for receiving and processing tissue and dispersing cells derived therefrom via a multi-channel spraying apparatus for use in the practice of delivering disaggregated cells to surfaces.

### BACKGROUND OF THE INVENTION

Numerous advantageous biological processes can be accelerated by seeding various biological surfaces and non-biological surface interfaces with biological surfaces with cellular material to accelerate appropriate growth and biocompatibility of that surface. For example, spontaneous closure and healing of large wounds may take extended periods of time during which infection and necrosis are common. Some wound healing methodologies used require transplanting significant amounts, e.g., sheets and flaps, of an individual's own skin to cover lesions. The difficulties inherent in this procedure are well-documented. More recently, cellular transplant technology has been used to promote healing in skin lesions.

With respect to an application of cells on a skin surface, several techniques have been described to apply epidermal cells to skin wounds. The application of cultured epidermal cells to wounds to improve healing has been well known to those skilled in the art since 1985. Autografts (made from an individual's own cells), cultured from small samples of skin, can be used to generate sheets of cells that can cover larger surfaces. Culturing skin cells on the large scale is a tedious and expensive process. Culturing autologous cells requires approximately two to four weeks from the time of harvest of cells to the time when an application of cell sheets can be made. These cultured sheets are fragile and difficult to handle and have a limited take (attachment). Cells are also being cultured together with various skin dressings.

Although a variety of systems and methods have been developed for harvesting tissue, and other systems have been developed for processing tissue, no system has been developed that provides for the sequential harvesting, processing and dispersing tissue in one operation. More specifically, no system has been developed that is self-contained and can be used anywhere, whether that is in an operating room or a military field hospital.

A critical component of the present invention is a device and means in which an individual's cells are mechanically and chemically disaggregated and used in resurfacing skin injuries. Delivered in the form of a spray, these cells are able to penetrate crevices of a wound or other surface giving rise to islands of confluent cells. Simultaneously applied adhesives serve as an attachment means for these disaggregated cells. This method of treatment is particularly useful for burn patients, particularly those that have limited skin available for transplantation. The present invention allows coverage of large areas of denuded flesh with cells that are obtained from a relatively small amount of healthy skin.

It is an object of this invention to provide a system for disaggregating tissue, processing such tissue, and spraying the disaggregated cells on a large surface area.

It is also an object of the present invention to provide a method for sterilely harvesting tissue, enzymatically and mechanically processing tissue, delivering cells and adhesives to a surface.

It is an object of the invention to provide a means for sterilely harvesting tissue, disaggregating such tissue, and storing such tissue until used.

Other objects, advantages and features of the invention will be readily apparent to one skilled in the art from the following detailed description hereof, taken in conjunction with the accompanying drawings.

### SUMMARY OF THE INVENTION

The present invention provides a system and method for harvesting and processing tissue and dispersing cells derived therefrom. The components of the invention include processing enzymes, means for holding processing enzymes, means for mechanically processing tissue, or means for enzymatically processing tissues, means for ensuring the sterile handling of tissue, means for simultaneously delivering cells and adhesives, means for storing processed tissue, and means for retaining the components in a sterile manner.

According to one embodiment of the invention, the means for simultaneously delivering cells and adhesives is a multi-channel spray apparatus designed for simultaneous delivery of skin cells and suitable adhesives to wounds and to dressing surfaces. One preferred embodiment of the present invention comprises a compressed air distribution compartment, a plurality of chambers and delivery tubes. An even more preferred embodiment additionally comprises a handle with steering mechanism. Another preferred embodiment additionally comprises a deflector cone. The compressed air distribution compartment comprises a hood and base. In one preferred embodiment the plurality of chambers includes a chamber for containing the disaggregated tissue in fluid and a chamber for containing an adhesive. Another embodiment additionally includes a chamber for containing an activator or other component of an adhesive. Each of the components is more fully described as follows.

COMPRESSED AIR DISTRIBUTION COMPARTMENT. This component preferably is made from a hard plastic material. It comprises a hood having openings for delivery tubes and an air flow inlet for a source of incoming compressed air. The hood is attached to the compartment's base component. The base component includes a means for attaching chambers containing fluids including a fluid containing the disaggregated cells of interest. In one preferred embodiment the means of attachment is a threaded opening that corresponds to threads presented by the chambers such that the chambers may be screwed to the compressed air distribution compartment. A bayonet-locking mechanism is also possible.

CHAMBERS. Preferably, the chambers are made of a clear material such as plastic or glass that have a means of attachment to the corresponding sites on the base of the compressed air distribution compartment. The tissue processing chambers allow the enzymatic tissue dissolution process and the cellular application process to run continuously without interruptions necessary for the tissue transfers. While the tissue and cells remain in one chamber, processing solutions can be changed sequentially during the entire process. After the tissue disaggregation process is complete, the tissue can be transferred into the tissue vials of the spray apparatus. The tissue processing chambers can be filled with a solution to facilitate the dispersion of cells and attached to the spray apparatus and thereby become part of the sprayer apparatus. In one preferred embodiment, tissue chambers are made of clear material. In yet a more preferred embodiment, the tissue chambers are made of a hard plastic material. The components of a preferred embodiment of the tissue processing chambers are shown in

FIGS. 1 and 2. These components can be assembled and disassembled as the tissue is processed.

There is a means of access of the compressed air into each chamber. Solution delivery tubes are made of material of sufficient flexibility to lie loosely and freely in a chamber's cavity.

DELIVERY TUBES. Delivery tubes preferably are made of thick medical grade silicone. From the firm and air-tight insertion made into the openings in the walls of the air distribution compartment, delivery tubes extend on one side into a chamber and on the other side are secured together and placed into a securely fitted fluid deflector cone. In a preferred embodiment the fluid deflector cone is made of a lightweight, translucent material such as plastic.

Each adhesive delivery tube features a fluid regulatory compression valve. Changing the pressure in the valve (by twisting) regulates the flow of fluids in the tubes.

The delivery tube through which disaggregated cells are dispersed preferably has a wider inside aperture diameter and a wider outside diameter than the other delivery tubes which may have substantially smaller aperture and external diameters. In one preferred embodiment, all the delivery tubes are inserted and adhered to the openings on the front wall of the compressed air compartment.

At the opening of the corresponding chamber, delivery tubes have an air influx valve integrated in the walls of the delivery tube. In one embodiment, the delivery tube through which disaggregated cells are transmitted preferably has three such valves. In one preferred embodiment, delivery tubes for adhesives or an activator have only one valve each.

SPRAYER HANDLE. In a preferred embodiment having a sprayer handle, the sprayer handle is positioned above the chamber containing the disaggregated cells and connected to the air distribution compartment. In one preferred embodiment, a suitably shaped cover, preferably rubber, makes the handle easy for an operator to grasp. The handle preferably contains a battery-powered stirrer.

DEFLECTOR CONE. In preferred embodiments containing this component, the deflector cone is made of a firm material, preferably a hard plastic. External portions of all delivery tubes and the open ends of such delivery tubes are inserted and securely fitted to the protective plastic cone. The cone deflects the spray containing disaggregated cells and adhesive material and any activator and prevents dissipation of the sprayed material.

The method of the present invention involves collecting tissue, collecting non-heparinized blood from which serum is separated to produce a 10% solution that can be used as the fluid vehicle for dispersing cells and rinsing tissue fragments. In some preferred embodiments the tissue fragments are mechanically reduced in sized with preferably scissors or scalpels, placing tissue fragments in a system the provides for enzymatic digestion which may be facilitated by applying warmth or a method to expose the enzymes to the tissue more completely, e.g., agitation, moving this disaggregated cells to the component of the system that allows for simultaneous dispersement with an adhesive, and dispersing cells resulting therefrom on the desired surface.

### BRIEF DESCRIPTION OF THE DRAWINGS

FIG. 1 presents an overall transparent view of the proposed sprayer so as to reveal the positioning of the back elements and inner compartments. The components are: compressed air distribution compartment; hood (1) base (1A), compressed air inlet (2), handle (3) with a steering apparatus (27), disaggregated cell chamber (4), disaggregated cell delivery tube (5), air influx valve (6) on the tissue delivery tube, first adhesive chamber (7), first adhesive delivery tube (8), first fluid flow valve (9), second adhesive delivery tube (11), second fluid flow valve (12), open-ended plastic cone (13) on the second adhesive delivery tube and open-ended plastic cone (14) for the first adhesive delivery tube. Open-ended silicone tubes (25) (26) from the disaggregated cells delivery tube deflector plastic cone (23).

FIG. 2 is an enlarged view of the air distribution compartment hood (1), with openings for the fluid tubes. The first opening (16) for the first adhesive tubes, the second opening (17) for the second adhesive delivery tube, and a third opening (18) for the disaggregated cell delivery tube (18).

FIG. 3 is a view of the base of the air distribution compartment (1A). Visible are threaded means of attachments for the first (19) and second (20) adhesive chambers and a threaded means of attachment for the disaggregated cells chamber (21) and the attachment for the sprayer handle (22).

FIG. 4 exhibits an enlarged view of the base of the compressed air distribution compartment (1A). All chambers (vials) (7), (10), (4), are securely attached through the corresponding fittings on the base of the compartment (19), (20), (21). On the upper portion of the base (1A) the first adhesive delivery tube (8) can be seen tunneled through the opening on the first fluid flow compression valve (9). The first adhesive delivery tube and the disaggregated cell delivery tube are depicted in the corresponding chambers. Three openings of the air influx valve (6) are visible on the upper portion of the disaggregated cell delivery tube (5).

A smaller single opening of the first air influx valve (31) is located on the first (8) adhesive delivery tube. On the opposing side a similar valve opening of smaller size (30) is made on the second (11) adhesive delivery tube. A small bore silicone tubing (32) is glued inside the distal chamber portion of the first (8) adhesive delivery tube.

FIG. 5 shows a dorsal view of the air distribution compartment (1) (1A). First (8) and second (11) adhesive delivery tubes are seen on the dorsal aspect of the compartment. Proximal to the compressed air compartment both the first and second tubes are inserted in to the first (9) and second (12) flow regulator valve. Externally both adhesive tubes are attached to the larger disaggregated cell delivery tube (5). Adhesive delivery tubes are tipped with narrow pointed cones (23), (24). The disaggregated cell delivery tube has an open end made of telescopically arranged thinner tubes (25) (26) inserted and glued into the larger main tubing (5).

FIG. 6 shows the frontal (external) arrangement of all tubes the first adhesive delivery tubes (8, 14), disaggregated cell delivery tubes (5, 25, 26) and the second adhesive delivery tubes (11, 13), and the deflector cone (23) in relation to other components of the cell delivery sprayer.

FIG. 7 is a dorsal transparent view through the hood (1) and the underlying base (1A) of the compressed air distribution compartment. An opening (21) for the disaggregated cell delivery tube is visible. Portions of the disaggregated cell delivery tube (5), portions of the first (8) and portions of the second (11) adhesive delivery tubes are also visible. Second (30) and first (31) air influx valves are seen on the respective adhesive delivery tubes. Immediately anterior to the hood and positioned on the top of the base (1A) there are first (9) and second (12) fluid flow regulatory valves. External portions of all fluid delivery tubes (8, 5, 11) immediately prior to their merger under the deflector cone (23).

FIG. 8 depicts a ventral aspect of the base (1A) with threaded attachments (21) for the disaggregated cell chamber and the attachment (20) for the second adhesive chamber. The attachment for the handle (22) is also depicted. Portions of the second (10) and the first (7) chambers are also present. A disaggregated cell delivery tube with openings for an air influx valve (6) is located on the disaggregated cell delivery tube. A small bore tube (32) is inserted into the second adhesive delivery tube.

FIG. 9 depicts the components of a preferred embodiment of the system of the present invention.

FIG. 10 depicts one preferred embodiment of the tissue processing chamber in a configuration to be attached to the spray system.

FIG. 11 shows an embodiment of the tissue processing chamber as comprising a chamber (1), a close fitting telescopic separator (4), a filter (5) attached to the distal end of the separator, a holder (2) and a stand (3).

FIG. 12A shows a preferred embodiment of the tissue processing container with before being pulled upwards to create a negative pressure gradient. FIG. 12B shows the same preferred embodiment after it has been pulled upwards and a negative pressure gradient applied across the filter holding tissue.

### DETAILED DESCRIPTION OF PREFERRED EMBODIMENTS

The system comprises a kit, which can be completely sterilized, preferably with ethylene oxide, and be available immediately for use in the operating room when required or, in an emergency, the components of the kit are: (1) a sprayer, (2) adhesive, (3) a means for mechanically separating tissue and cells from tissues; (4) a means for enzymatically processing cells from tissue; (5) a means for ensuring a sterile environment for tissue and cell harvesting processing; (6) a means for warming tissue and solutions; and (7) a means for retaining the components in a safe and stable fashion.

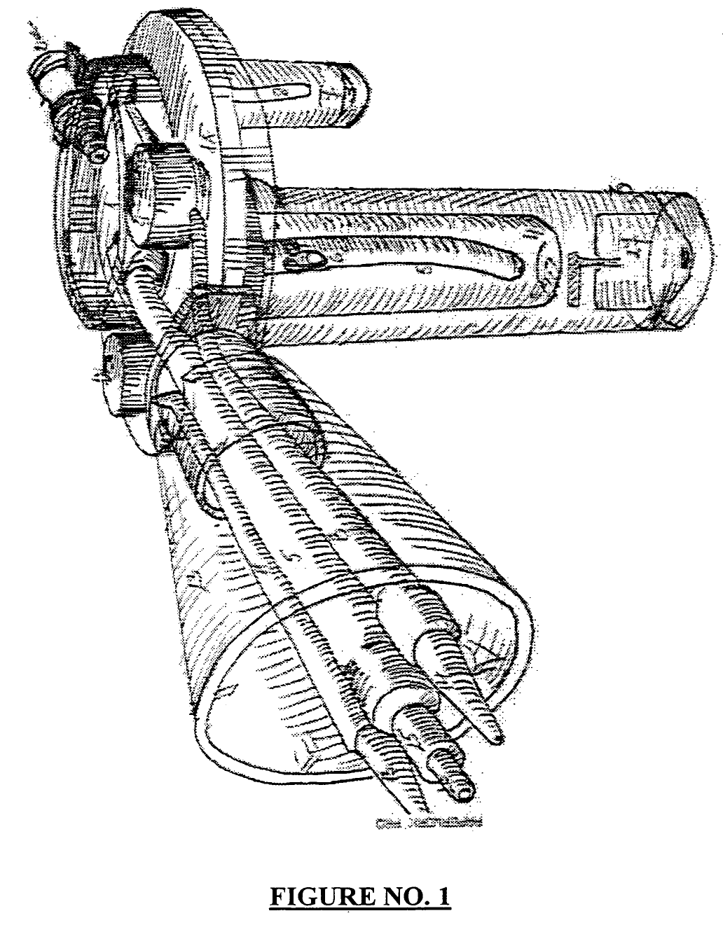

The sprayer of the present invention, as illustrated in the FIG. 1, preferably is constructed of a hard medical grade plastic material (e.g., polyurethane), and of pliable medical grade silicone tubes. Lightweight elements, preferably plastic, are assembled and adhered together, preferably with an adhesive (epoxy), while silicone tubes are adhered with a silicone adhesive. Chambers are also made of medical grade plastics (polyurethane) or Pyrex glass materials. The steering mechanism preferably is made of a stainless steel material and a magnet.

Referring to the figures, the present invention has a fluid outflow system that includes centrally positioned disaggregated cell·delivery tube (5) which preferably has a cross-sectional diameter which is substantially longer than that of the adhesive delivery tubes (8), (11).

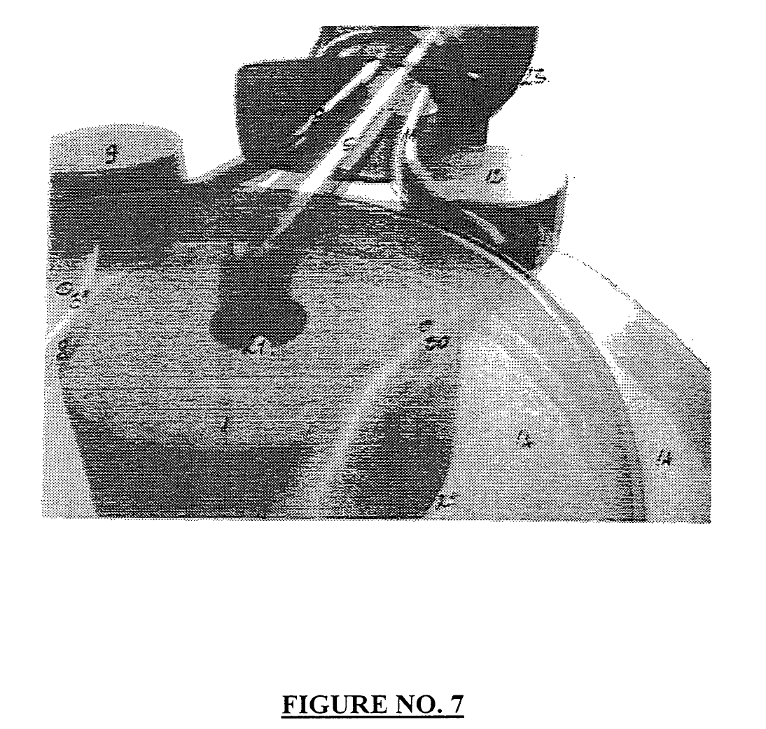

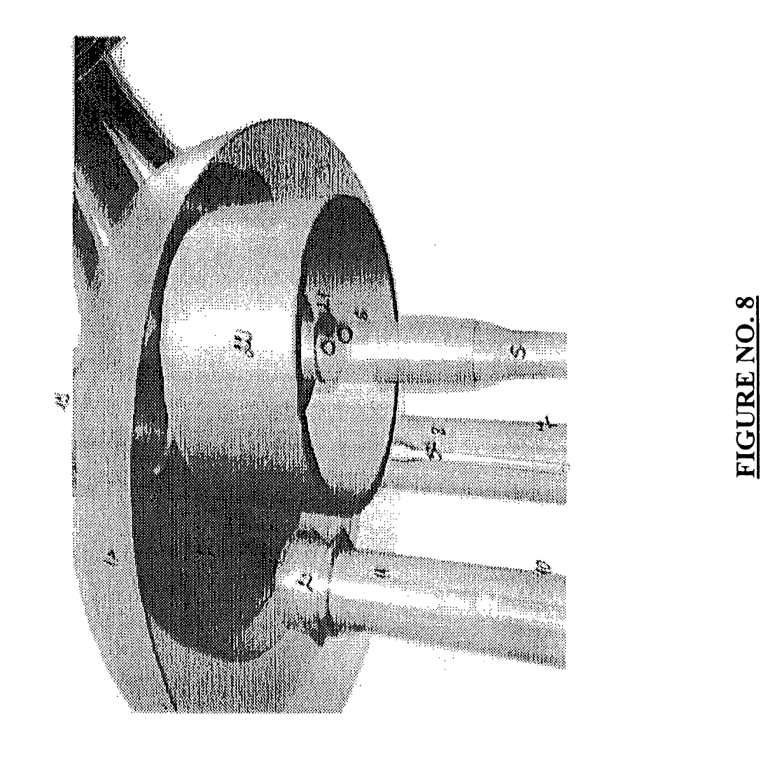

A preferred embodiment of the invention has two firmly bonded points of attachment. The external one preferably is achieved through the use of a silicone adhesive or any other suitable bonding adhesive. The disaggregated cell conducting tube is glued on each side to the corresponding adhesive conducting tubes (FIGS. 5, 6 and 8). The other fixation point is done by securely fitting all fluid conducting tubes into respective openings made on the hood of the compressed air compartment. As further depicted in FIGS. 1, 4, 7 and 8, the inside and outside diameters of the disaggregated cell delivery tube are greater than that of the adhesive delivery tubes. Also shown on FIGS. 1, 4 and 8 is that all fluid tubes are free distally from their stabilization point at the openings made on the hood of the compressed air chamber. Silicone tubes serving as fluid conducting tubes have a necessary degree of flexibility and are loosely and freely movable within the lumen of corresponding attached chambers. The length of each fluid delivery tube should be such that the distal opening of tubes lies comfortably within the chamber without touching the bottom of the chambers. Immediately after exiting the external portions of the adhesive delivery tubes are fitted fluid flow valves.

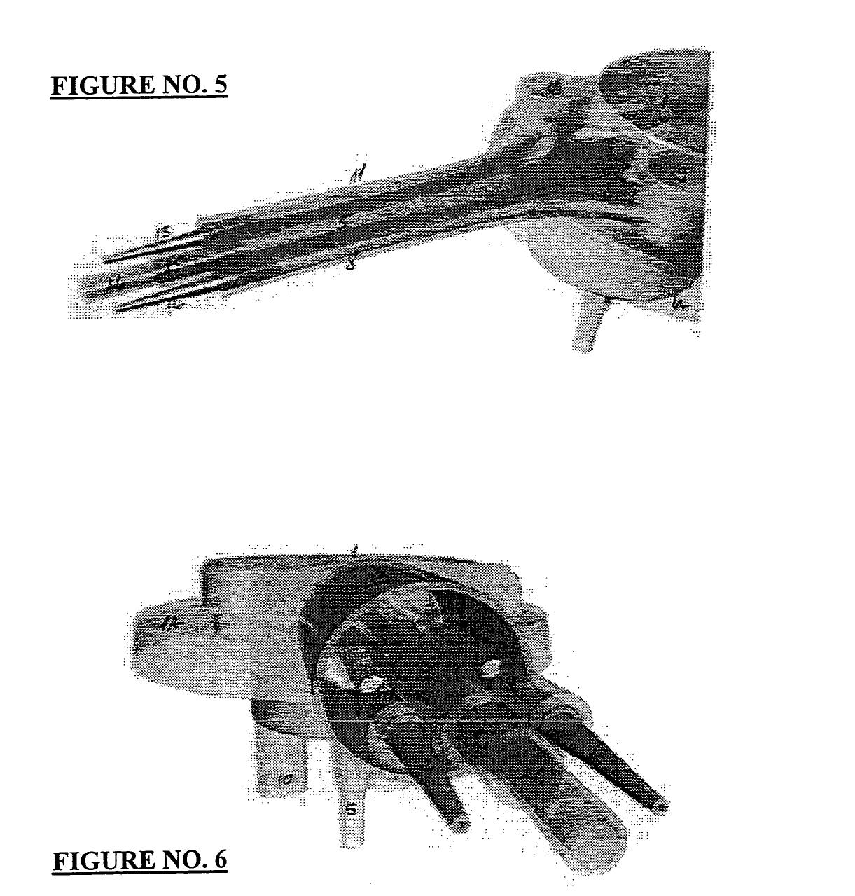

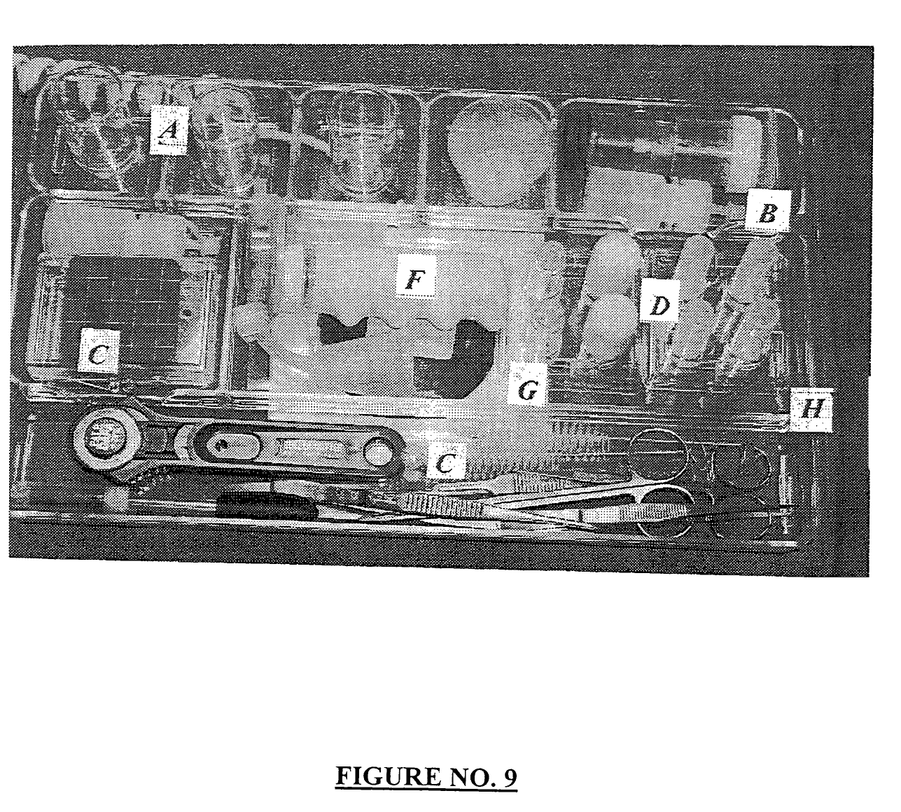

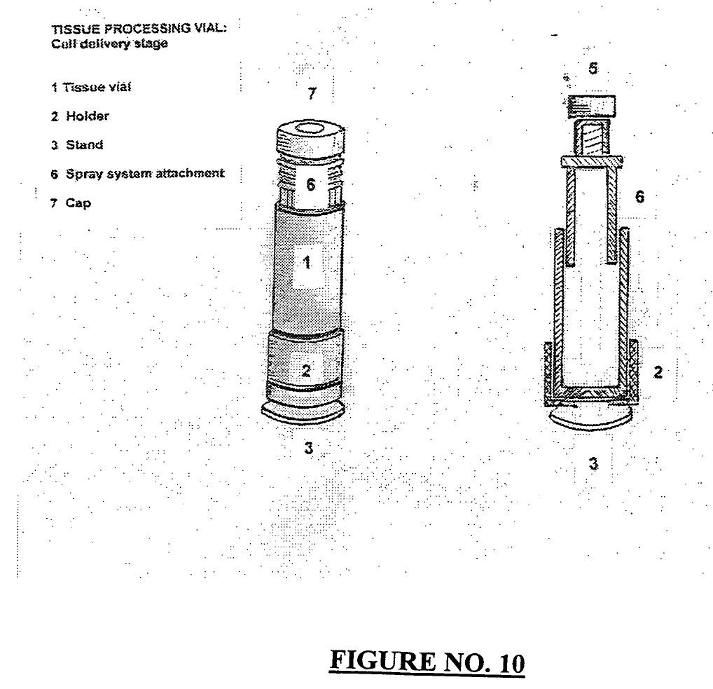

As depicted in FIGS. 4 and 8, a small piece of tubing featuring a smaller inside and outside diameter is inserted and adhered in the distal portion of the first adhesive delivering tube. Distally from the fixation point at the hood openings, FIGS. 1, 7 and 8 show a plurality of perforations (in the preferred embodiment depicted herein, three) (6) on the disaggregated cell delivery tube, and a single perforation on each of the adhesive delivery tubes. These are innovative air influx valves that have perforations of approximately 12G (gauge) to 14G (gauge) in the disaggregated cell tube, 18G in the first adhesive tube and 16G in the wall of the second adhesive tube.

This valve allows a portion of the compressed air in the corresponding chambers to be transferred into the path of the propelled fluids in the fluid delivery tubes giving rise to an even low-pressure spray. This mechanism creates an internally produced spray that allows an open-ended spray system instead of a closed system typically known to those skilled in the art. Due to the open-end system, larger conglomerations of disaggregated cells, tissue particles, and fluids of different consistency, can be delivered in form of a spray.

The present invention is substantially different from currently available spray systems in which the size and the shape of spray components are regulated by the mechanism at the spray exiting point.

The air distribution system preferably is made of medical grade plastic materials (e.g., polyurethane). It preferably is composed of a transparent hood, preferably plastic, and a substantially flat base, preferably plastic, that is larger in circumference than the hood. The hood is attached to the flat base, preferably through an adhesive such as glue. On its frontal portion, the hood has openings for at least one fluid delivery tube, and an opening for the compressed air inlet. Although the fitting between the fluid delivery tubes and these openings is secure, the compressed air inlet is adhered to the corresponding opening on the opposite side of the hood.

The base on its external portion has threaded attachments for chambers and the handle of the sprayer. These attachments are positioned within the perimeter of the hood and open into the hood space. When the chambers are attached, there is a continuous open space between the chambers and the air distribution compartment, which is partially filled with the delivery tubes.

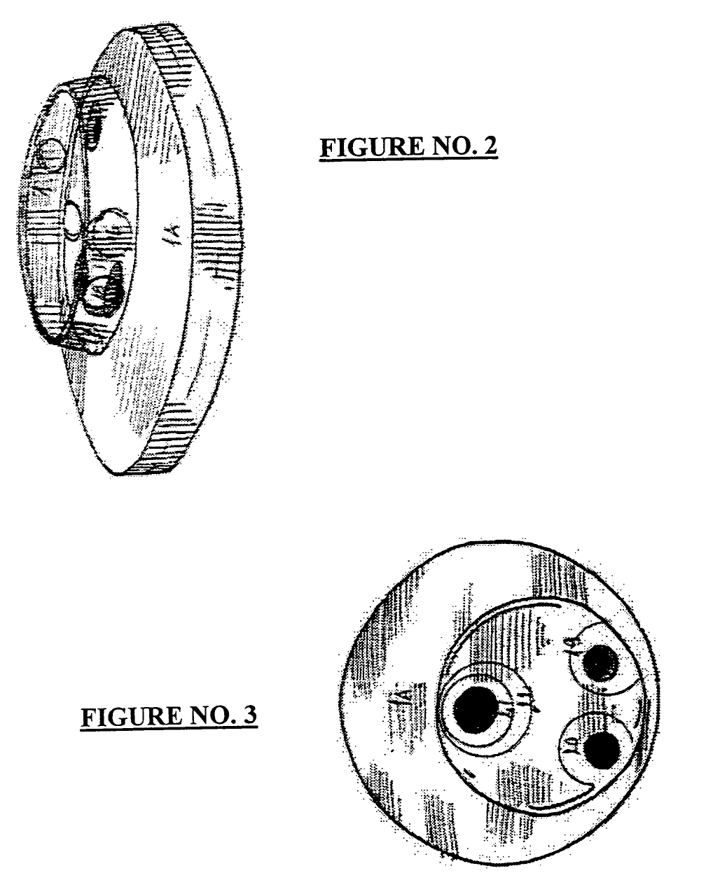

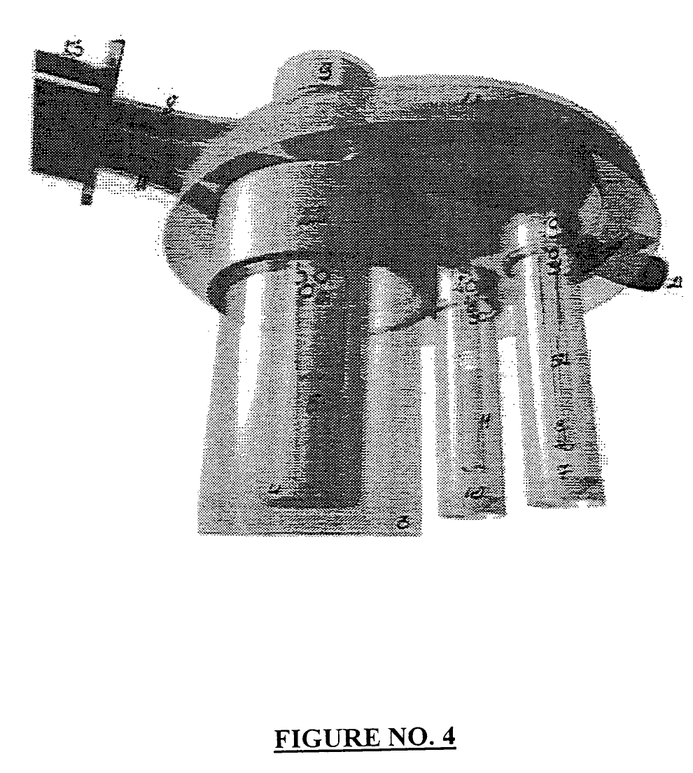

The disaggregated cell chamber preferably is made of a disposable Pyrex glass material. The adhesive chambers preferably are made of a disposable polyurethane material. All chambers are threaded at the openings to engage the threaded apertures at the base of the air distribution compartment. In one preferred embodiment, tissue chambers are made of clear material. In yet a more preferred embodiment, the tissue chambers are made of a hard plastic material. The components of a preferred embodiment of the tissue processing chambers are shown in FIGS. 1 and 2. These components can be assembled and disassembled as the tissue is processed.

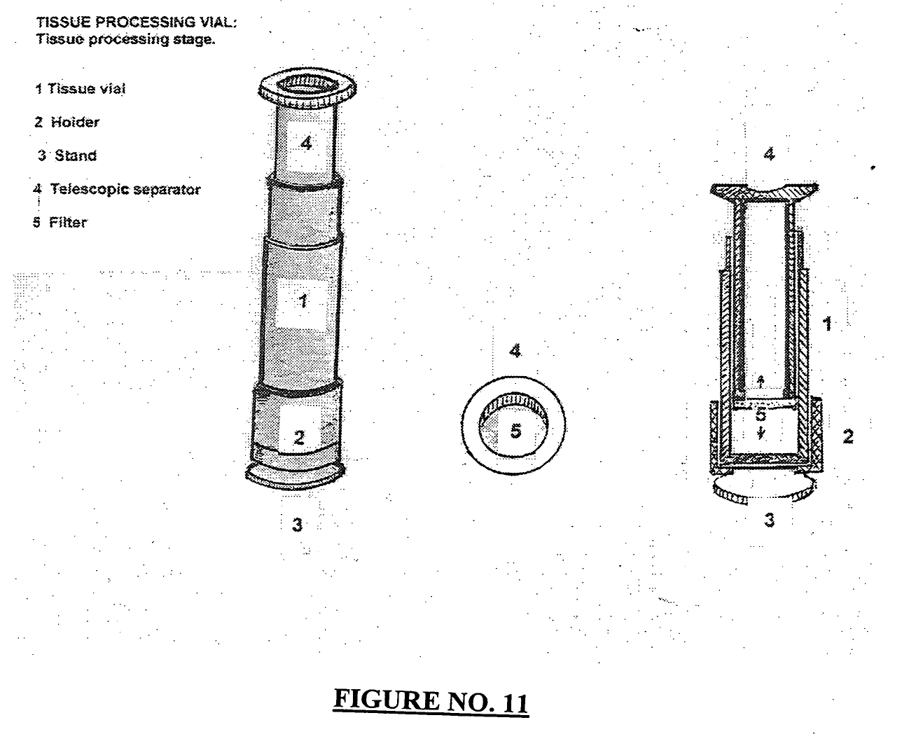

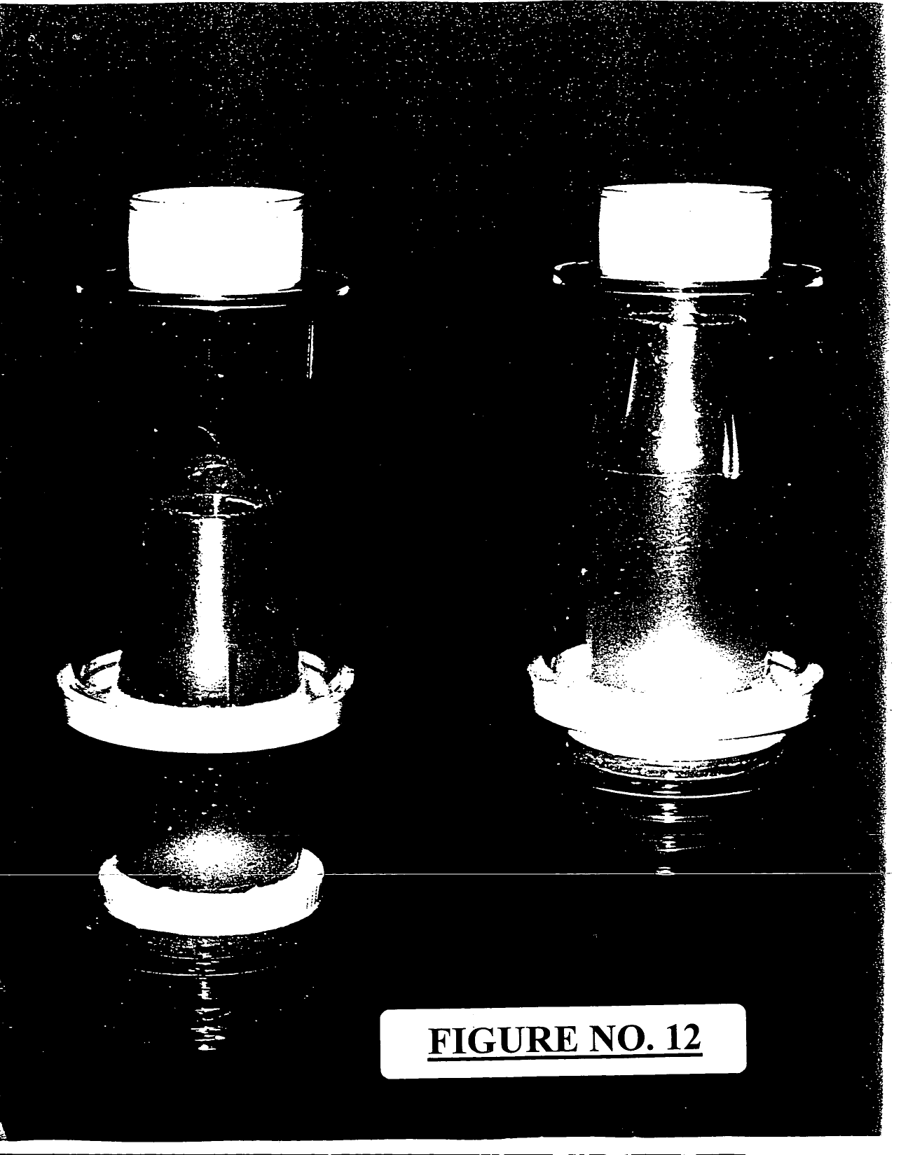

In one preferred embodiment, shown in FIG. 11, the tissue processing chamber is comprised of a chamber (1), a close fitting telescopic separator (4), a filter (5) attached to the distal end of the separator and a holder (2). In one preferred embodiment the processing chamber also includes a stand (3). FIG. 10 depicts one preferred embodiment of the tissue processing chamber in a configuration to be attached to the spray system. FIG. 10 depicts a chamber (1), a means for engaging the spray system (6), a holder (2), a stand (3), and a removable cap (7). In this embodiment, the telescopic separator (4) has been removed after completion of the tissue processing, and the means for attaching to the spray system (6) is inserted in to the tissue chamber (1).

The present invention illustrated and described herein in detail is not limited to skin cell delivery. It is understood that the present invention may disseminate a variety of cells and solutions.

In one application, disaggregated cells are disseminated to a wound surface or dressing surface. The present invention facilitates the dissemination of cells of interest, and their attachment (through the inclusion of the sprayed adhesives) to various surfaces, biological and non-biological.

Due to the multiple delivery capabilities, the present invention can be used for creating completely new dressings by simultaneously delivering cells and dissolved active substances to the new dressing. The dressing will then prime the wound surface when applied to the wound.

The system of the present invention also has a means of retaining the components of the system in a manner that will ensure their retention and is compatible with the needs of a variety of environments. In one preferred embodiment, the components are housed in and on a compartmentalized lightweight tray, which even more preferably is made of a durable plastic material.

The system of the present invention preferably has a thermal means of warming tissue and accelerating chemical processes. In one preferred embodiment the thermal means is a heating pad.

The system of the present invention also has a means for handling and mechanically disaggregating tissue. Such means may include one or more from the following group of elements: a tissue cutting board, syringes, scalpel handle, forceps, scissors, needles and scalpel blades.

The present invention also has a means of enzymatically processing tissue which includes a tissue processing chamber and at least one vial containing at least one enzyme (e.g., dispase and trypsin) and may include one of the group of a portable tissue stirrer or pipettes and pipette bulbs. In one preferred embodiment processing enzymes are applied sequentially.

The present invention also has a means of ensuring the sterility of the tissue handling. In one preferred 7 embodiment the system has a surgical cap, a surgical mask and surgical gloves for a system user to don before handling the tissue and disaggregated cells and sterile towels.

According to the method of the present invention, tissue is collected and rinsed. Depending on the embodiment of the invention and the size of the tissue collected, they may be mechanically reduced in size so that they can be accommodated by the vials of the system of the present invention. Such mechanical disaggregation may be accomplished by a variety of means provided by the system of the present invention including scissors or scalpels. The resulting tissue fragments are then inserted in the tissue processing chamber of the present invention along with digestive enzymes. The action of the digestive enzymes of the present invention can be accelerated or facilitated through a variety of means including agitation by shaking, bubbling (preferably through a bulb which is a component of the device of the present invention) or thermal means (in one preferred embodiment a heating pad), depending on the embodiment of the invention.

According to one embodiment of the method of the present invention that uses a tissue chamber having the features described above, the telescopic separator is detached from the tissue chamber. Tissue is fragmented and transferred into the tissue chamber. Enzymatic solutions are poured into the chamber. The telescopic separator with a filter attached to its distal end can be inserted into the tissue chamber until it reaches the level of the enzymatic solution in the chamber. In one preferred embodiment of the method, the chamber containing tissue and enzymatic solution, compressed with the separator, is agitated. In another preferred embodiment, the tissue-solution combination is additionally or alternatively heated to accelerate the tissue dissolution process. In one preferred embodiment such heating is achieved through application of the warming pad.

After the tissue has achieved a desired level of disassociation, the telescopic separator can be pushed deeper in to the chamber until it gently compresses the tissue on the bottom of the chamber. The filter on the end of the telescopic separator is permeable to the solution in the chamber, but impermeable to tissue and cells. The solution is removed and the telescopic separator is moved upward. Depending on whether further processing of the tissue and cells is desired, the steps of adding solution, bringing the separator down to the fluid level, optionally agitating and heating the tissue-solution combination, compressing the tissue and cells, and removing the effluent, can be repeated as many times as is necessary or desired.

After the tissue and cells have been processed according to the needed specifications, the telescopic separator is removed and the means for engaging the spray system is inserted firmly into the chamber. A spray solution is added to the cellular material in the chamber. The chamber is then attached to the spray apparatus.

Another embodiment of the tissue processing container comprises a chamber having a septum with an aperture for a rigid cylinder with a tapering open-ended cone at one end. See FIGS. 12A and 12B. The rigid cylinder is inserted into the chamber such that its outer circumference is securely engaged with the aperture in the septum and the tapered open-ended cone extends into the chamber. The other end of the rigid cylinder has a means for securely engaging a tissue restraining cylinder. The tissue restraining cylinder has both a means to engage the rigid cylinder and to engage a cap to prevent the spillage of its contents. A filter, which is permeable to fluids when pressure is applied, but impermeable to tissue and cells, is inserted between the cylinder at the end that has a means for engaging the tissue restraining cylinder and the tissue restraining cylinder. The exterior bottom of the tissue processing container has a means for engaging a secure surface, preferably a threaded means which provides for twist-on engagement. The tissue restraining cylinder further has a cap which engages with the tissue restraining cylinder.

In this embodiment, when the conical end of the rigid cylinder is inserted into the chamber, tissue is placed inside the tissue restraining cylinder and on the filter. A processing solution is applied to the tissue within the tissue restraining cylinder. When the tissue is being bathed by the processing solution, no pressure exists across the filter and thus the processing solution remains in the tissue restraining cylinder. See FIG. 12A. When the tissue has had sufficient exposure to the processing solution, as determined by the protocol and users needs, the rigid cylinder is pulled upwards without disengaging it from the chamber. See FIG. 12B. This action creates negative pressure in the chamber compelling the processing solution to pass through the filter into the chamber while the tissue and cells remain on the other side of the filter. When the processing solution has been sufficiently evacuated from the tissue restraining cylinder into the chamber, the rigid cylinder can be reinserted into the chamber and a new solution can be applied to the tissue. Alternatively, if the tissue processing is complete, tissue and cells can be transferred from the filter to the spray vial. Cells that remain on the filter can be disengaged by pulling the rigid filter upward, applying solution within the tissue restraining cylinder, pushing the tissue restraining cylinder back into the chamber, thereby applying pressure which will disengage cells trapped on the filter. The now suspended cells can be added to cells in the spray vial. The process for removing trapped cells can repeated as necessary. During the foregoing procedures, the cap may be applied to the tissue restraining cylinder and the cylinder may be agitated or the chamber may remain stationary by engagement of the means of attachment on its exterior bottom to another surface.

According to another embodiment of the method of the present invention that does not use the multi-faceted tissue chambers described above, after the tissue has been processed in one chamber, the resulting cells are transferred, using pipettes and or syringes of the present invention to the tissue vials of the sprayer. In one preferred embodiment non-heparinized blood is collected from the host organism, which is used to produce a 10% solution that can be used as the fluid vehicle for dispersing cells. The disaggregated cells and the solution are combined in the container of the sprayer. A means of supplying compressed air is engaged with the sprayer and applied such that the disaggregated cells in solution are dispersed on the desired surface.

Although preferred embodiments of the invention have been described herein in detail, it is understood by those skilled in the art, that variations may be made thereto without departing from the spirit of the invention or the scope of the appended claims.

An Example of a Preferred Embodiment

A system and method for harvesting and processing tissue and dispersing cells which includes a multi-channel spray device for use in disseminating cells to wound surfaces or wound dressings, wherein said sprayer is comprised of an air distribution compartment, at least one delivery tube, preferably a plurality of delivery tubes, and even more preferably three delivery tubes, at least one chamber, preferably a plurality of chambers and even more preferably a plurality of chambers, and a handle containing a steering, or aiming, mechanism which preferably is rubber covered. Wherein, said compressed air distribution compartment has a means to allow distribution of compressed air through all the chambers of the sprayer. Said compressed air distribution compartment has a non-interrupted continuous open space with chambers attached to the base of the compartment. Even more preferably, the disaggregated cell delivery tube has greater diameter and thickness than the adhesive delivery tubes. Even more preferably, in the invention, the delivery tubes are firmly attached to the openings on the hood of the compressed air distribution compartment. In a more preferred embodiment, the delivery tubes are free distally to the fixation point. Even more preferably, the delivery tubes external to the fixation point are adhered to each other. In one embodiment, the invention has a fluid deflector, preferably such fluid deflector is made of a light weight material such as plastic, even more preferably wherein such material is transparent, wherein the fluid deflector covers the delivery tubes externally to their engagement with the hood. In one embodiment, all the delivery tubes have an open end. The delivery tubes are not obstructed at any point. Further, each delivery tube has a distal free end positioned in the chamber cavity. The delivery tubes preferably are made of medical grade silicone with sufficient flexibility to permit said tubes to lie loosely and freely in the chamber cavities without touching the bottom of the chambers. The fluid delivery tubes have air influx valves positioned on the upper portion of their chamber segments. The air influx valve diverts a portion of the compressed air from the chambers into the path of fluid in the delivery tubes.

The present invention further comprises a sprayer that has a plurality of chambers, and even more preferably one featuring three chambers. Even more preferably the chamber storing disaggregated cells is made of Pyrex glass. The chambers storing an adhesive component is preferably made of polyurethane plastics. The chambers are engaged in the position on the base of the compressed air distribution compartment.

The present invention provides for a kit that can be used in the operating room or other surgical setting (e.g., physician office, clinic, field office) which allows all of the processes of skin preparation and cell fluid carriers to be used in one surgical operation.

1. A system for receiving, processing and dispersing cells comprising:
(a) a multi-channel sprayer device comprised of
(i) at least one chamber;
(ii) at least one delivery tube having an air influx valve for each said chamber;
(iii) a handle having an aiming mechanism;
(iv) a compressed air distribution compartment having a means to allow distribution of compressed air through said chamber and non-interrupted continuous open space with said chamber attached to the base of said compartment;
(b) processing enzymes;
(c) containers for said processing enzymes;
(d) a means for storing tissue;
(e) a means for mechanically separating tissue and cells from tissue;
(f) a biologic adhesive;
(g) a means for sterile handling of tissue and cells;
(h) a means for warming tissue and solutions;
(i) a means for retaining components a-h in a sterile fashion; and
(j) a source of compressed air.

2. The system of claim 1, wherein said sprayer has at least two chambers wherein a first chamber contains disaggregated tissue and cells and a second chamber has said biologic adhesive.

3. The system of claim 2, wherein said sprayer has a third chamber containing an activator for said biologic adhesive.

4. The system of claim 1, wherein said sprayer further comprises a powered stirrer in said handle.

5. The system of claim 1, further comprising a deflector cone.

6. The system of claim 1, wherein said means for mechanically separating tissue and cells from tissue is at least one from the group of scissors and scalpel.

7. The system of claim 1, further comprising a telescopic separator having a filter on a distal end which is permeable to fluids but impermeable to tissue and cells.

8. The system of claim 1, further comprising:
i. a chamber having a septum having an opening for a rigid plastic cylinder;
ii. a rigid cylinder having an outer diameter sufficiently large such that the rigid cylinder is movably engaged within said opening wherein a first end of said cylinder is housed within said chamber and said second end is external to said chamber and has a means to engage a means for retaining tissue across the opening of said second end;
iii. a filter which is permeable to fluids but impermeable to tissue and fluids, covers the area of said second end, and is inserted between said rigid cylinder and said means for retaining tissue; and
iv. said means for retaining tissue which engages with said rigid cylinder.

9. The system of claim 1, wherein said means for heating tissue and solutions is a heating pad.

10. The system of claim 1, wherein said means for sterile handling of tissues and cells is a pair of sterile gloves, a surgical mask, and a surgical cap and may further include any of the group of forceps, needle, sterile towel and tissue cutting board.

11. A method for processing and dispersing cells comprising the steps:
(a) harvesting tissue;
(b) rinsing said tissue;
(c) mechanically disaggregating said tissue into multiple units;
(d) placing the product of step c in a container;
(e) applying at least one digestive enzyme to said product of step c;
(f) placing the product of step e in a solution;
(g) placing the product of step f in a system that provides for simultaneous disbursement of cells and adhesives using compressed air; and
(h) applying compressed air to said system.

12. The method of claim 11, wherein step e further comprises agitating said digestive enzyme and said product of step c.

13. The method of claims 11 and 12, wherein step e further comprises heating said digestive enzyme and said product of step c.

14. The method of claim 11, wherein step e is repeated at least once.

15. The method of claim 11, wherein step d further comprises extending a telescopic separator having a filter on the end extended into said container wherein said filter is permeable to fluids but impermeable to tissue and cells into said container, such that said filter of said telescopic separator compresses said product of step c between the interior walls of said container and said filter.

16. The method of claim 11 wherein said container is a cylinder which engages a second cylinder, wherein said second cylinder extends through and is movably engages an aperture in a septum over a chamber such that a first end of said second cylinder is within said chamber and a second end of said cylinder engages said first cylinder and presents a filter that permeable to fluids but impermeable to tissue and cells, such that said filter spans the opening where said first cylinder and said second cylinder are engaged; and wherein step e further comprising moving said second filter upward out of the chamber such that a negative pressure is applied across said filter and said digestive enzyme is drawn through the filter into the chamber.

17. A device for receiving and dispersing cells comprising:
(a) a multi-channel sprayer device comprised of:
i. a first chamber for holding cells in solution;
ii. a second chamber for holding a biologic adhesive;
iii. at least one delivery tube having an air influx valve for said first and second chambers;
iv. a handle having an aiming mechanism;
v. a compressed air distribution compartment having a means to allow distribution of compressed air through said first and chambers and non-interrupted continuous open space with said first and second chambers attached to the base of said compartment;
(b) a biologic adhesive; and
(c) a source of compressed air.

18. The system of claim 19, wherein said sprayer further comprises a third chamber for holding an activator for said biologic adhesive wherein said biologic adhesive require activation, at least one delivery tube having an air influx valve for said third chamber, and wherein said compressed air distribution compartment further has a means to allow distribution of compressed air through said third chamber attached to said base of said compartment.
